(19) United States
(12) Patent Application Publication
(10) Pub. No.: US 2012/0157816 A1
(43) Pub. Date: Jun. 21, 2012

(54) ELECTROCARDIOGRAM SIGNAL SENSOR WITH A WAISTBAND

(76) Inventor: CHIN-YEH HUNG, TAOYUAN COUNTY (TW)

(21) Appl. No.: 12/971,441

(22) Filed: Dec. 17, 2010

(51) Int. Cl. A61B 5/0402

(52) U.S. Cl. 600/382

(57) ABSTRACT

An electrocardiogram signal sensor with a waistband wherein the waistband is partially made of insulating material and placed on the waist. A positive electrode and a negative electrode facing to the left and right sides of the waist of the user's waist is exposed on the internal surface of the waistband. The positive and negative electrodes are extended forward by a positive lead and a negative lead, respectively to establish an electric connection to a transmitter at the front side of the waistband. In this way, the positive and negative electrodes at both sides of the internal surface of the waistband are in contact with both sides of the user's waist for monitoring the voltage frequency change when the blood flows through the user's waist.

### BACKGROUND OF THE INVENTION

1. Field of the Invention

The invention relates to an electrocardiogram signal sensor, and more particularly, to a waistband type sensor in which the positive and negative detection electrodes are in contact with both sides (flanks) of the waist for obtaining the optimal signals of the voltage frequency change.

2. Description of the Related Art

Conventionally, people's heart rate remarkably varies before and after people take exercise. During exercise, measuring the variables is very important. To take exercise properly and advantageously, the heart rate must increase to 60% of the maximum rate; however, if the heart rate increases to the maximum rate during exercise, it will do harm to the exerciser. Thus, a user-friendly and precise heart rate measurement device is very important for taking exercise to keep fit.

Next, FIG. 1 shows an electrode belt of heart rate monitor disclosed in U.S. Pat. No. 6,553,247, in which a heart rate monitoring unit 60 is placed on the chest with a belt 70 to monitor the heart rate on and on and wirelessly transmit a signal to a receiver 80 placed around the wrist. However, it is found that the patent is poor with the following conditions when being applied.

1. Generally, the heart rate monitoring unit 60 is tightly bound with the belt 70 on the chest to keep close thereto; however, the lung capacity increases during exercise and thus the belt bound up tightly on the chest makes a user uncomfortable and breathe hard.

2. Female chest contour is bigger so that the heart rate monitoring unit 60 cannot stay fully close to the chest, thereby decreasing the monitoring accuracy.

3. To accurately measure the heart rate, the heart rate monitoring unit 60 cannot be placed outside the clothes but inside the clothes and close to the chest. Therefore, the belt 70 can be bound in place only after the clothes is taken off, which might be somewhat inconvenient for males but may be implemented none the less. However, it is very inconvenient for females to bind up the belt 70 in a public place; thus, the conventional belt is not suitable for women in taking exercise.

### SUMMARY OF THE INVENTION

A primary object of the invention is to eliminate the above-mentioned drawbacks and to provide an electrocardiogram signal sensor with a waistband that is placed on the user's waist like a belt or waistband. This electrocardiogram signal detection way is completely free from the influence of the clothing or the female breast. The use of the electrocardiogram signal sensor in accordance with the invention is ergonomic, convenient and without any burden. Moreover, the detection electrodes are closely in contact with both sides of the user's waist, thereby improving the detection accuracy. Especially, it is suitable for women in taking exercise.

Another object of the invention is to provide an electrocardiogram signal sensor with a waistband wherein the positive and negative electrodes are connected when the blood flows through the user's waist. The voltage will be generated during the blood circulation. A more accurate detection of the heart rate is available by the fact that the frequency of the voltage change is not the same when the heart rate is different and by the fact that the more obvious is the change of the voltage frequency, the farther the blood of a person is spaced apart.

In order to achieve the above-mentioned objects, an electrocardiogram signal sensor includes a waistband that is partially made of insulating material and placed on the waist. A positive electrode and a negative electrode facing to the left and right sides of the waist of the user's waist are exposed on the internal surface of the waistband. The positive and negative electrodes are extended forward by a positive lead and a negative lead, respectively to establish an electric connection to a transmitter at the front side of the waistband.

The transmitter includes an electrocardiogram processing circuit to which the positive and negative leads are electrically connected. The electrocardiogram signals received by the transmitter will be first processed and then transmitted to a microcontroller. The electrocardiogram signals can be stored in the microcontroller or transmitted by a high-frequency transmitting unit to a receiving unit at the periphery of the waistband.

In this way, the positive and negative electrodes at both sides of the internal surface of the waistband are in contact with both sides of the user's waist for monitoring the voltage frequency change when the blood flows through the user's waist. After amplified by the electrocardiogram processing circuit, the signals are processed by the microcontroller to determine the value of the electrocardiogram signal of the user.

### BRIEF DESCRIPTION OF THE FIGURES

The accomplishment of this and other objects of the invention will become apparent from the following descriptions and its accompanying figures of which:

FIG. 1 is a schematic drawing of an electrode belt of a heart rate monitor, the electrode belt according to U.S. Pat. No. 6,553,247;

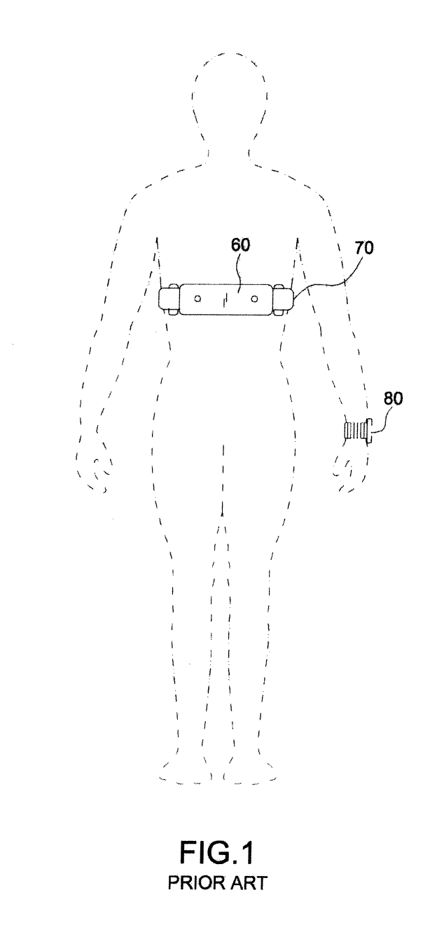

FIG. 2 is a perspective view of a preferred embodiment of the invention seen from the front side thereof;

FIG. 3 is a perspective view of the preferred embodiment of the invention seen from the rear side thereof;

FIG. 4 is a block diagram of a circuit according to the invention;

FIG. 5 is an application view of the invention; and

FIG. 6 is another application view of the invention.

### DETAILED DESCRIPTION OF THE PREFERRED EMBODIMENT

Now, the present invention will be described more specifically with reference to the following embodiments. It is to be noted that the following descriptions of preferred embodiments of the invention are presented herein for purpose of illustration and description only; it is not intended to be exhaustive or to be limited to the precise form disclosed.

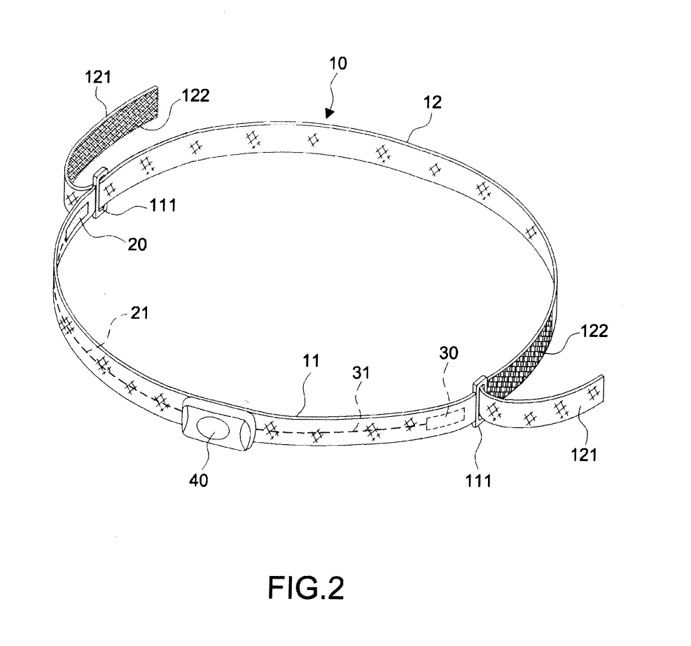

First, with reference to FIGS. 2 through 4, the invention includes a waistband 10. The waistband 10 is partially made of insulating material and can be placed on the waist. A positive electrode 20 and a negative electrode 30 facing to the left and right sides of the waist of the user's waist are exposed on the internal surface of the waistband 10. The positive and negative electrodes 20, 30 are extended forward by a positive lead 21 and a negative lead 31, respectively to establish an electric connection to a transmitter 40 at the front side of the waistband 10.

The transmitter 40 includes an electrocardiogram processing circuit 41 to which the positive and negative leads 21, 31 are electrically connected. The electrocardiogram signals received by the transmitter 40 will be first processed and then transmitted to a microcontroller 42. The electrocardiogram signals can be stored in the microcontroller 42 or transmitted by a high-frequency transmitting unit 43 to a receiving unit 50 at the periphery of the waistband 10.

Based on the above-mentioned configuration, the positive and negative electrodes 20, 30 at both sides of the internal surface of the waistband 10 are in contact with both sides of the user's waist for monitoring the voltage frequency change when the blood flows through the user's waist. After amplified by the electrocardiogram processing circuit 41, the signals are processed by the microcontroller 42 to determine the value of the electrocardiogram signal of the user.

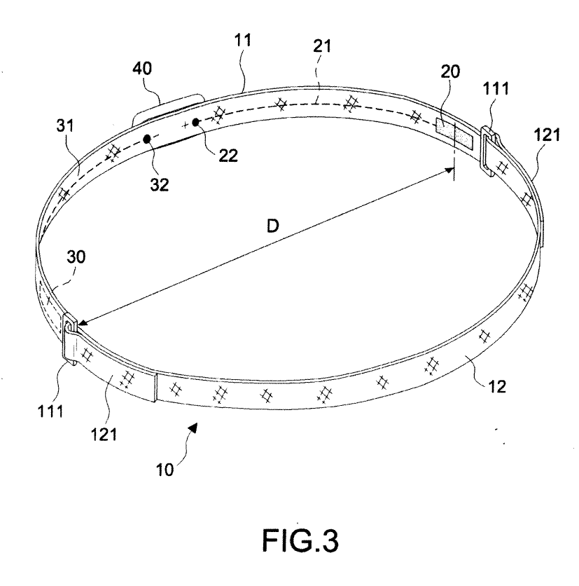

According to the preferred embodiment of the invention, the positive electrode 20 and the negative electrode 30 are positioned at the corresponding sides of the internal surface of the waistband 10. As shown in FIG. 3, they are positioned at the places where the internal diameter D of the waistband 10 is maximal. This design allows the positive and negative electrodes 20, 30 to be in contact with the user's waist so as to establish the connection. Meanwhile, the both sides of the waist has the maximal distance. The voltage will be generated during the blood circulation. A more accurate detection of the heart rate is available by the fact that the frequency of the voltage change is not the same when the heart rate is different and by the fact that the more obvious is the change of the voltage frequency, the farther the blood of a person is spaced apart.

According to the above-mentioned embodiment, the waistband 10 consists of a first band body 11 and a second band body 12, but should not be limited thereto. The first band body 11 is made of insulating material and has at both ends thereof connecting elements 111 for joining to both end portions 121 of the second band body 12. In this way, the waistband 10 is created. Moreover, the positive and negative electrodes 20, 30 are exposed on the internal surface of the first band body 11.

According to the embodiment, the positive and negative leads 21, 31 are made of conductive string and hidden within the first band body 11. Moreover, the positive and negative leads 21, 31 include at the front end thereof a positive and a negative terminals 22, 32 for establishing an electric connection to the transmitter 40 at the front side of the first band body 11.

In addition, the positive and negative leads 21, 31 can be made of conductive fabrics (not shown) and positioned on the internal surface of the first band body 11. Moreover, the positive and negative leads 21, 31 includes at the front end thereof a positive and a negative terminals 22, 32 for establishing an electric connection to the transmitter 40 at the front side of the first band body 11.

Besides, the positive and negative electrodes 20, 30 are selected from a group consisting of conductive metal piece, conductive fabrics, and conductive rubber.

The connecting elements 111 at both ends of the first band body 11 are constructed as retaining ring while both end portions 121 of the second band body 12 pass through the retaining ring and are pulled back to fix together in place by the Velcro-type fastening means 122.

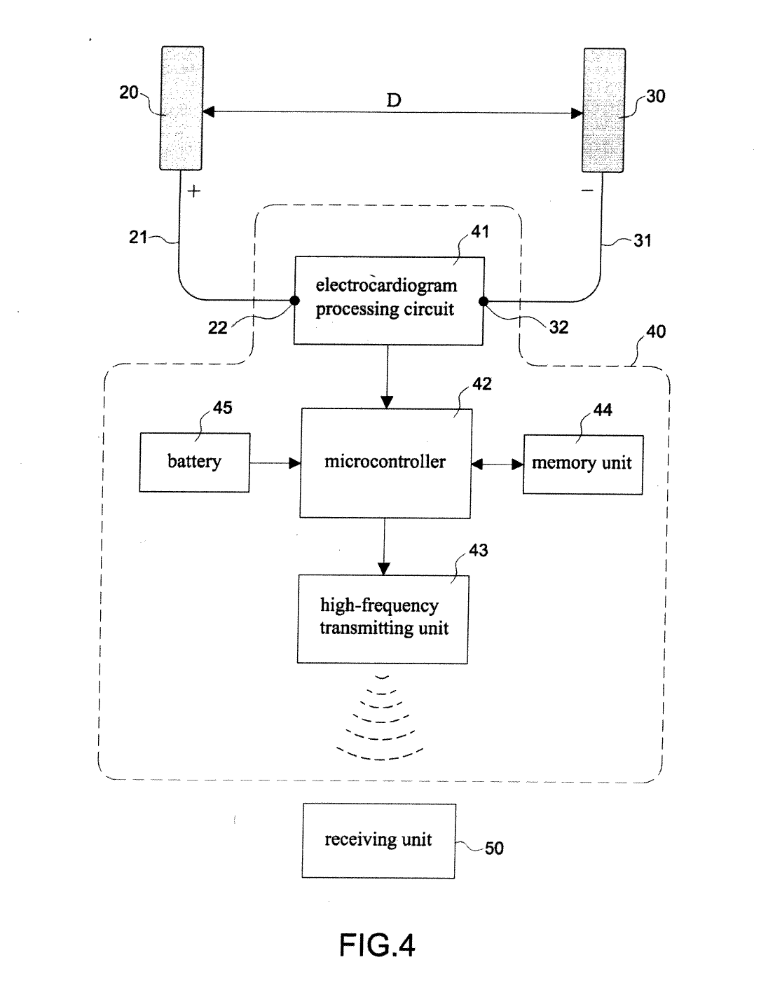

As shown in FIG. 4, the transmitter 40 includes internally a memory unit 44 for storing the data of the microcontroller 42 and a battery 45 for supplying the required power.

The receiving unit 50 for receiving the signals transmitted by the transmitter 40 is selected from a group consisting of watches, consoles, mobile communication equipments (e.g. mobile phone, PDA, NB, etc.) that are provided with a display.

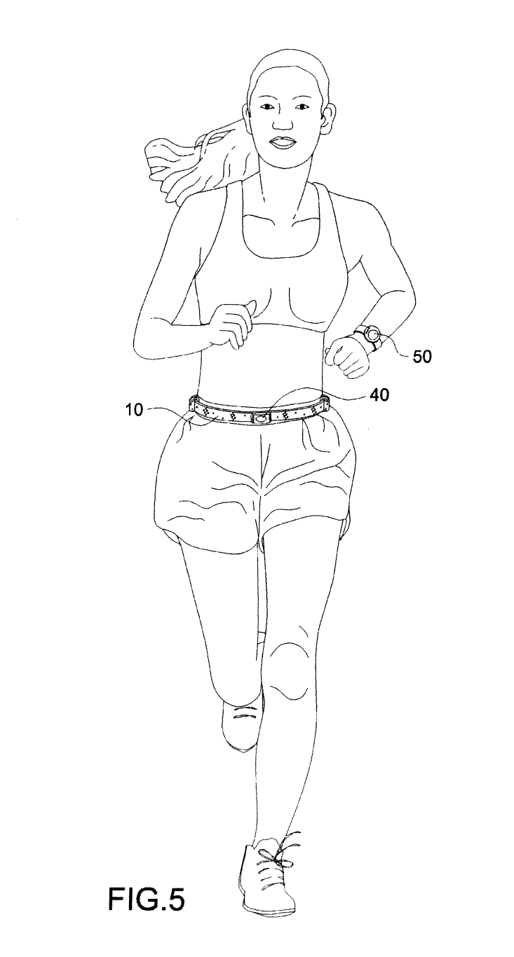

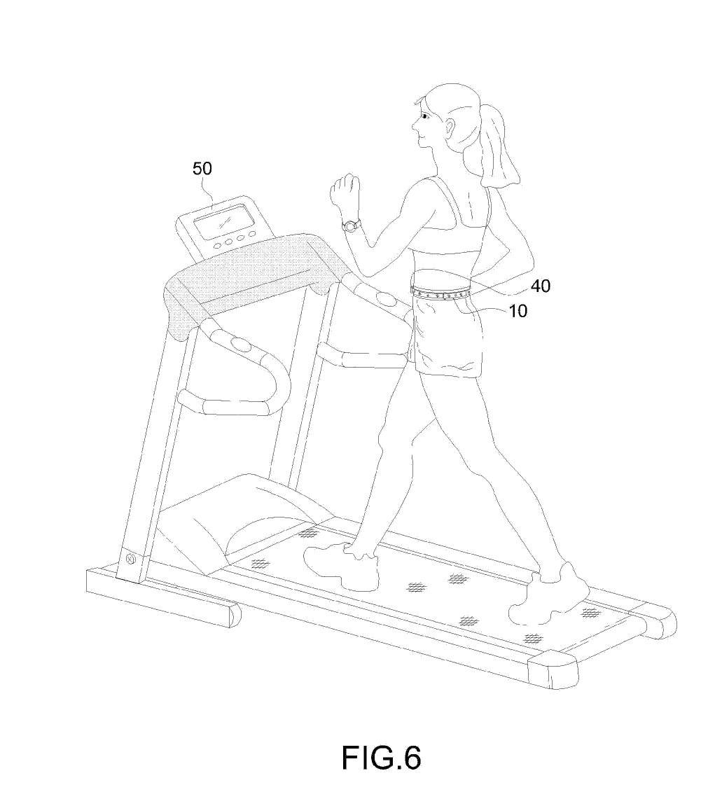

FIG. 5 illustrates an application example of the invention. The waistband 10 is bound on the waist. As a result, the positive and negative electrodes 20, 30 at both sides of the waistband 10 are located on both farthest sides of the body, thereby obtaining the accurate electrocardiogram signals. According to experimental results of the invention, the electrocardiogram signals obtained by use of the detection through electrodes at both sides of the waist are no less favorable than the signals obtained from the chest. In addition, the invention has advantages that the electrode belt of heart rate monitor of U.S. Pat. No. 6,553,247 does not have. That is, the present invention is easy to use and is especially suitable for women in taking exercise. According to this embodiment, the detected signals are transmitted by the transmitter 40 to the receiving unit 50 in form of watch. As shown in FIG. 6, the signals are transmitted to the receiving unit 50 in form of a console on a gymnastic apparatus.

According to the invention, the electrocardiogram signals are detected on the waist, thereby avoiding the disadvantages of the electrode belt of heart rate monitor of U.S. Pat. No. 6,553,247. Accordingly, the invention ensures the ergonomic and convenient use.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An electrocardiogram signal sensor with a waistband,
wherein the waistband is partially made of insulating material and placed on the waist, and a positive electrode and a negative electrode facing to the left and right sides of the waist of the user's waist are exposed on the internal surface of the waistband, and the positive and negative electrodes are extended forward by a positive lead and a negative lead, respectively to establish an electric connection to a transmitter at the front side of the waistband;
wherein the transmitter includes an electrocardiogram processing circuit to which the positive and negative leads are electrically connected, and the electrocardiogram signals received by the transmitter will be first processed and then transmitted to a microcontroller, and the electrocardiogram signals can be stored in the microcontroller or transmitted by a high-frequency transmitting unit to a receiving unit at the periphery of the waistband,
whereby the positive and negative electrodes at both sides of the internal surface of the waistband are in contact with both sides of the user's waist for monitoring the voltage frequency change when the blood flows through the user's waist, and after amplified by the electrocardiogram processing circuit, the signals are processed by the microcontroller to determine the value of the electrocardiogram signal of the user.

2. The electrocardiogram signal sensor with a waistband as recited in claim 1, wherein the positive electrode and the negative electrode are positioned at the places where the internal diameter of the waistband is maximal.

3. The electrocardiogram signal sensor with a waistband as recited in claim 1, wherein the waistband consists of a first band body and a second band body;
wherein the first band body is made of insulating material and has at both ends thereof connecting elements for joining to both end portions of the second band body, thereby creating the waistband; and
wherein the positive and negative electrodes are exposed on the internal surface of the first band body.

4. The electrocardiogram signal sensor with a waistband as recited in claim 3, wherein the positive and negative leads are made of conductive string and hidden within the first band body, and wherein the positive and negative leads include at the front end thereof a positive and a negative terminals for establishing an electric connection to the transmitter at the front side of the first band body.

5. The electrocardiogram signal sensor with a waistband as recited in claim 3, wherein the positive and negative leads are made of conductive fabrics and positioned on the internal surface of the first band body, and wherein the positive and negative leads includes at the front end thereof a positive and a negative terminals for establishing an electric connection to the transmitter at the front side of the first band body.

6. The electrocardiogram signal sensor with a waistband as recited in claim 3, wherein the positive and negative electrodes are selected from a group consisting of conductive metal piece, conductive fabrics, and conductive rubber.

7. The electrocardiogram signal sensor with a waistband as recited in claim 3, wherein the connecting elements at both ends of the first band body are constructed as retaining ring while both end portions of the second band body pass through the retaining ring and are pulled back to fix together in place by the Velcro-type fastening means.

8. The electrocardiogram signal sensor with a waistband as recited in claim 1, wherein the transmitter includes internally a memory unit and a battery.

9. The electrocardiogram signal sensor with a waistband as recited in claim 1, wherein the receiving unit for receiving the signals transmitted by the transmitter is selected from a group consisting of watches, consoles, mobile communication equipments that are provided with a display.
